(19) United States
(12) Patent Application Publication
(10) Pub. No.: US 2009/0285763 A1
(43) Pub. Date: Nov. 19, 2009

(54) INFANT AEROSOL DRUG DELIVERY SYSTEMS AND METHODS

(76) Inventors: Warren H. Finlay, Edmonton (CA); John Storey-Bishoff, Edmonton (CA); Michelle Noga, Edmonton (CA)

Correspondence Address: Lambert Intellectual Property Law, Suite 200, 10328 - 81 Avenue, Edmonton, AB, T6E 1X2, CA

(21) Appl. No.: 12/465,488

(22) Filed: May 13, 2009

Related U.S. Application Data
(60) Provisional application No. 61/052,736, filed on May 13, 2008.

(51) Int. Cl. A61K 9/12; A61M 16/10; A61B 5/107

(52) U.S. Cl. 424/43; 128/203.14; 600/587

(57) ABSTRACT

A delivery system and method for delivering an aerosol drug to an infant. The delivery device comprises a) a diagnostic module configured to provide geometrical properties of the nasal airway of the infant as output; b) a dosing system configured to produce an aerosol drug dose, based upon the output of the diagnostic module, that is predicted to ensure that a desired amount of the aerosol drug dose reaches the lungs of the infant in use; and c) an infant facemask connected to receive the aerosol drug dose from the dosing system for supply of the aerosol drug dose to the infant.

### CROSS-REFERENCE TO RELATED APPLICATIONS

This application claims the benefit under 35 USC 119(e) of U.S. provisional patent application No. 61/052,736 filed May 13, 2008.

### TECHNICAL FIELD

This document relates to infant aerosol drug delivery systems and methods.

### BACKGROUND

Aerosol-based drugs consist of an air suspension of solid and/or liquid particles that are commonly used for the therapeutic treatment of lung diseases as well as other diseases. The most frequently used aerosol-based drug delivery systems/devices include pressurized metered dose inhalers (pMDIs), dry powder inhalers (DPIs) and nebulizers. Optimal particle sizes delivered by these devices are in the range of 1-5 microns in order to achieve optimal clinical benefit since larger particles land in the mouth and are swallowed, while smaller particles tend to be exhaled and much smaller particles are difficult to deliver in sufficient mass.

MDIs are used to administer bronchodilators, anti-inflammatory agents and steroids. The output of an MDI typically ranges from 20 μg to 5 mg. However, lung deposition of the administered drug is estimated to be approximately 10-50% for most MDIs on average, with the remainder of the drug depositing in the mouth and throat.

DPIs generate aerosol by passing air through a dose of dry powder medication. The powder medication is either in the form of micronized drug particles or micronized drug particles that are bound to carrier particles which yields agglomerates. Inspiration flow draws the particles and then deagglomerates them into drug particles that target the lung while the carrier particles deposit in the mouth. The magnitude and duration of the patient's inspiratory flow influences aerosol generation from a DPI. DPIs have a high mouth-throat deposition of approximately 50% and low lung deposition of around 20-50%.

Nebulizers produce mist (aerosol) that is inhaled through a mouthpiece or mask. The lung deposition associated with some nebulizers is approximately 10-40% with mouth-throat deposition around 10-20%, and device deposition and wastage making up the remainder.

Presently, the doses are prescribed based primarily on the infant's weight and severity of disease. However, the amount of the inhaled aerosol drug that will be deposited in the lungs of a particular infant cannot be predicted with reasonable accuracy because there is large variability in the amount of the aerosol drug that is deposited in the nasal airway region of different individuals. In fact, it is typical that a relatively small amount of the inhaled drug reaches the targeted region (i.e. the lungs) since a large portion of the inhaled drug deposits in the nasal airway region where it can create adverse local effects.

Existing inhalers typically can provide an active pharmaceutical ingredient in a single amount, such as 200 mcg or 400 mcg for example. A medical practitioner can then prescribe an inhaler that provides a 200 mcg amount with enough medication for 100 doses. However, for a 200 mcg dose amount, the lungs of one individual may only receive 50% of the dose while the lungs of another individual may only receive 20% of the dose. In actual fact, the amount of drug deposition in the lungs can vary between 10 to 90% amongst different infants, for example. This is not dangerous for medication that has a wide therapeutic window. However, for aerosol-based drugs with more advanced molecules, for example inhaled insulin, the medical practitioner must be very careful with the prescribed dose because if too much of the medication reaches the lungs then the individual will overdose and possibly suffer severe adverse consequences or even death. Conversely, if not enough of the medication reaches the lungs then, the therapeutic effect is not sufficient and the individual's physical condition will not be improved, or may worsen. Therefore, for aerosol-based drugs with narrow therapeutic windows, it is quite important to be sure of the amount of the drug that is deposited in the lungs of the individual.

A large number of nasal studies exist in the literature where the focus is on adults or older children. For example, in vivo adult studies and in vivo studies of both adults and children have been performed. A study that compares deposition in casts of a monkey nasal airway to that in an adult human airway has also been done. Recent computational fluid dynamics nasal modeling work highlights the importance of accurate turbulence modeling in the nose, and looks at inter-species differences in particle deposition in rats, monkeys and humans.

The published data does not allow a straightforward predictive, quantitative understanding of nasal deposition in infants.

### SUMMARY

A method is disclosed of determining the dose of an aerosol drug to be delivered to an infant comprising: a) determining a desired amount of the aerosol drug to be delivered to the lungs of the infant; b) obtaining values for the geometrical properties of the nasal airway of the infant; and c) determining a dose of the aerosol drug, according to the geometrical properties of the nasal airway, that will deliver the desired amount of the aerosol drug to the lungs of the infant.

A delivery system is also disclosed for delivering an aerosol drug to an infant, the delivery device comprising: a) a diagnostic module configured to provide geometrical properties of the nasal airway of the infant as output; b) a dosing system configured to produce an aerosol drug dose, based upon the output of the diagnostic module, that is predicted to ensure that a desired amount of the aerosol drug dose reaches the lungs of the infant in use; and c) an infant facemask connected to receive the aerosol drug dose from the dosing system for supply of the aerosol drug dose to the infant.

In some embodiments, step c) further comprises generating nasal deposition prediction data based on a Stokes number that depends on a length scale D based on the geometrical properties of the nasal airway of the infant. In further embodiments, the nasal deposition prediction data is further based on a Reynolds number that depends on a length scale D based on the geometrical properties of the nasal airway of the infant. In even further embodiments, the nasal deposition prediction data is further based on a parametric correction that depends on a length scale D based on the geometrical properties of the nasal airway of the infant. In some of the above-indicated embodiments, one or more of the length scale D of the Stokes number, the length scale D of the Reynolds number, and the length scale D of the parametric correction is based upon a mean diameter of the nasal airway, for example if D=V/Aₛ.

These and other aspects of the device and method are set out in the claims, which are incorporated here by reference.

### BRIEF DESCRIPTION OF THE FIGURES

Embodiments will now be described with reference to the figures, which are not to scale and in which like reference characters denote like elements, by way of example, and in which:

FIGS. 1A-1K are perspective views of models of the nasal airway passages of ten different infants, as well as a purchased model. Subjects 2-8 are illustrated in FIGS. 1A-1G, respectively, the purchased model is illustrated in FIG. 1H, and subjects 10, 11, and 14 are illustrated in FIGS. 1J, 1K, and 1I, respectively. These models are the models studied by John Storey-Bishoff, et al. in “Deposition of micrometer-sized aerosol particles . . . ”, Journal of Aerosol Science, 39 (2008), pp 1055-1065.

FIG. 2 is a flow diagram of an experimental setup used in the study.

FIG. 3 is a graph that illustrates deposition vs. impaction parameter (standard error too small to display).

FIG. 4 is a graph that illustrates deposition vs. Stk (standard error too small to display).

FIG. 5 is a graph that illustrates deposition vs. f (Re, Stk) (standard error too small to display).

FIG. 6 is a graph that illustrates normalized pressure drop vs. Reynolds number.

FIG. 7 is a graph that illustrates deposition vs. non-dimensional parameter D=V/AS (standard error too small to display).

FIG. 8 is a graph that illustrates Deposition vs. non-dimensionalized parameter based on √(V/L) (standard error too small to display).

FIG. 9A is a block diagram of an exemplary inhaler that is used in accordance with the embodiments disclosed herein.

FIG. 9B is a block diagram of another exemplary embodiment of an inhaler in accordance with the embodiments disclosed herein.

FIG. 10 is a diagram of another exemplary embodiment of an inhaler in accordance with the embodiments disclosed herein.

FIG. 11 is a diagram that illustrates a delivery device.

FIG. 12 is a flow diagram that illustrates a method of determining the dose of an aerosol drug to be delivered to an infant.

### DETAILED DESCRIPTION

Immaterial modifications may be made to the embodiments described here without departing from what is covered by the claims.

Nomenclature

$$Re = Reynolds number ( \frac{\overline{Q} ρ}{D μ} )$$

$$Stk = Stokes Number ( \frac{\overline{Q} {ρ}_{particle} {d}^{2} Cc}{18 μ {D}^{3}} )$$

D=length scale based on geometric properties of the nasal airway

D_{avg}=length

Q̅=average volumetric flow rate

V=airway volume

Aₛ=airway surface

D_{avg}=average value of D for all subjects studied

d=particle diameter

Cc=Cunningham slip factor (1+2.52 λ/d)

λ=is the fluid mean free path

ρ=fluid density

μ=fluid viscosity

ρ_{particle}=particle density

η=fraction not passed through replica

L=airway length

Infants less than approximately 4 to 6 months of age are obligate nasal breathers since this facilitates breathing while breast feeding. Nose breathing is also common during low activity at all ages. For this reason, as well as reasons of compliance and practicality, face masks are used when administering pharmaceutical aerosols to infants. The nasal path is therefore important in determining lung drug dose for this age group. The filtering function of the nose is also important when an infant is exposed to environmental aerosols since it prevents some particles from entering the lung.

In vivo measurement of aerosol deposition with infants is difficult due to issues of compliance, legitimate concerns over safety, and the non-voluntary nature of participation of the infant. In vitro studies based on anatomical models of infant nasal geometries overcome most of these concerns and a limited number of such models have been constructed by other researchers.

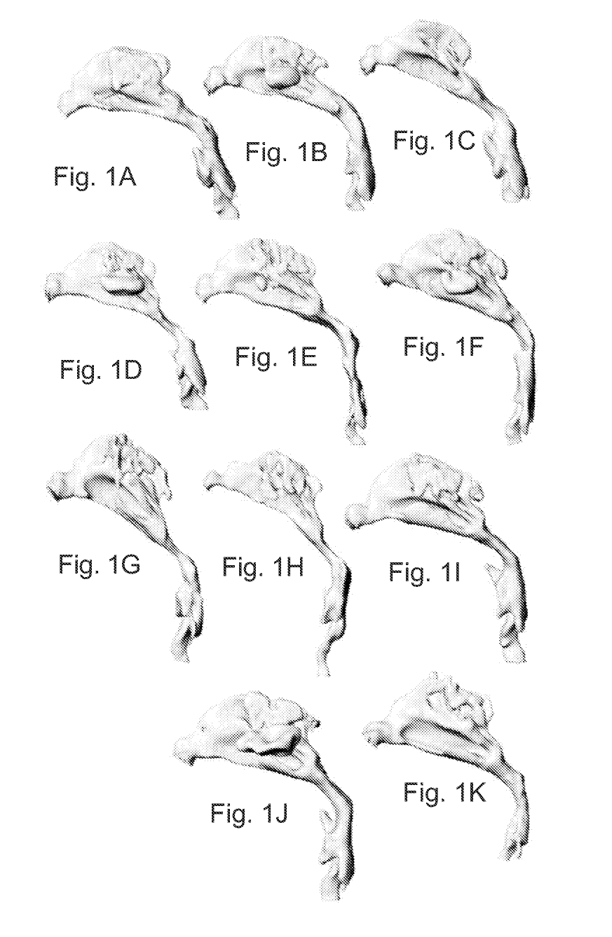

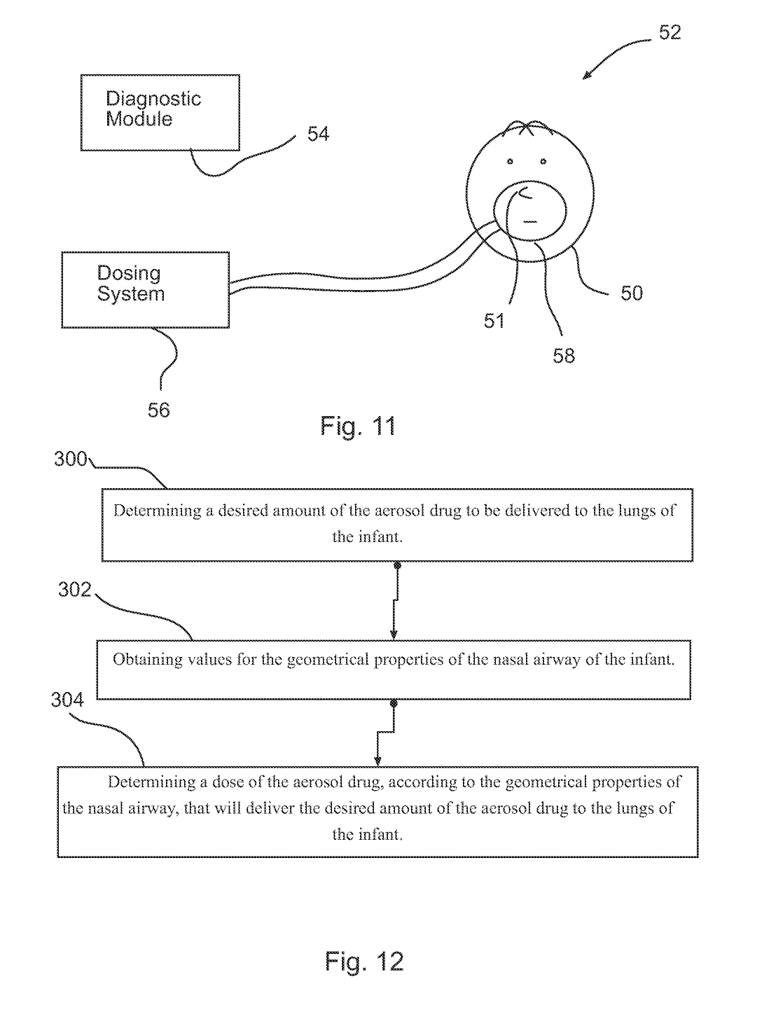

Referring to FIG. 11, a delivery system 52 for delivering an aerosol drug to an infant 50 is illustrated. The delivery device 52 comprises a diagnostic module 54, a dosing system 56, and an infant facemask 58. The diagnostic module 54 is configured to provide geometrical properties of the nasal airway (shown in FIGS. 1A-1K) of the infant 52 as output. The diagnostic module may be a CT scanner, an acoustic rhinometry system, a rhinomanometry system, a magnetic resonance imager (MRI) or other suitable systems such as any medical imaging system. The diagnostic module 54 need not be connected directly or indirectly to the dosing system. In some embodiments, the diagnostic module 54 is in a separate room or facility.

The dosing system 56 is configured to produce an aerosol drug dose based upon the output of the diagnostic module 54 to ensure that a desired amount of the aerosol drug dose reaches the lungs of the infant 50 in use.

The infant facemask 58 is connected to receive the aerosol drug dose from the dosing system 56 for supply of the aerosol drug dose to the infant 50. Aerosol delivery described herein to an infant requires at least a delivery to the nose 51 of the infant 50. Infant facemask 58 may partially (as shown) or fully cover the face of the infant 50.

Referring to FIG. 12, a method of determining the dose of an aerosol drug to be delivered to an infant is illustrated. Referring to FIG. 11, in a stage 300 (shown in FIG. 12), a desired amount of the aerosol drug to be delivered to the lungs of the infant 50 is determined. This may be done by a medical practitioner after diagnosing the infant 50 with a condition requiring aerosol drug treatment. In a stage 302, (shown in FIG. 12), values are obtained for the geometrical properties of the nasal airway (shown in FIGS. 1A-1K) of the infant 50. This may be done using the diagnostic module 54. It should be understood that stage 302 may be carried out before stage 300, for example if the geometrical properties are taken from previous tests carried out on the infant. In a stage 304, a dose of the aerosol drug is determined, according to the geometrical properties of the nasal airway, that will deliver the desired amount of the aerosol drug to the lungs of the infant 50. The method may further comprise delivering the dose of the aerosol drug to the infant 50.

The embodiments of the apparatus and method disclosed in this document are discussed alongside an investigation of ten infant nasal replicas and a purchased infant nasal airway replica as described by Janssens et al. in “The sophia anatomical infant nose . . . ”, Journal of Aerosol Medicine, 14, 433-441, 2001, hereinafter “Janssens”) is presented. The purchased infant nasal airway replica (hereinafter referred to as subject 15) is of a 9 month old infant, and was purchased from Erasmus M C, Rotterdam, Netherlands. Details of the construction of this model can be found in Janssens, which is incorporated herein by reference.

A correlation which predicts nasal deposition in these 11 nasal replicas for micrometer-sized aerosol particles based on geometry Stokes, Reynolds and Stokes, and Reynolds, Stokes, and a length scale number were then constructed which closely matches the measured data and allows quantitative prediction of infant nasal aerosol deposition.

The study carried out used data from computed tomography (CT) scans of infants scanned for medical purposes. This is one way that stage 302 could be carried out. All subjects were deemed to have normal nasal structure and the original CT scans were not acquired because of any nasal or sinus problem. All scans were obtained with the patient in the supine position. Imaging was helical with reconstructed axial slices of 1.25 mm thickness and in plane resolution ranging from 291 to 430 μm across subjects.

The airways were identified in the CT based on gray level using the Mimics software package (Materialise, Ann Arbor, Mich.). An upper threshold of approximately −295 Hounsfield units was used. All airways extended from the nares to just past the larynx. Sinuses were kept if they were connected to the airway in the Mimics model. The airways were smoothed to eliminate surface roughness due to noise and discretization in the CT data. The area of the face from chin to forehead and including both cheeks was also identified in the CT data. Using the Magics software package (Materialise) these geometries were used to create models which could be built using a rapid proto-typer (Invision SR 3-D printer from 3D Systems, Rock Hill, S.C.). The models were built in two parts with the face and throat built separately and later joined with bolts and sealed externally with putty. The build material was acrylic plastic and a wax support material was used. The support material was melted to expose the completed model segments. The parameters of the infant nasal airways were calculated and detailed in Table 1 below. All models were subsequently CT scanned and a comparison was made of the airway parameters listed in Table 1 between the original subjects and the built models. Volume differed on average by 5.28%, airway surface by 5.24%, minimum cross-sectional area by 3.93%, the computed parameter D differed by 7.85% (tending to be larger in the models than the original CT data), while length remained unchanged. This indicates that the models accurately represent the subjects' original nasal CT geometries and also that the error involved in model construction is small compared to the inter-subject variability of these parameters. In Table 1 below, V is the volume of the nasal airway, Aₛ is the surface area of airway lumen, L is a representative path length through the model, Aₘᵢₙ is the minimum cross-sectional airway taken perpendicular to expected airflow, D is the calculated dimension V/Aₛ.

TABLE 1
Parameters of the infant nasal airways

| | Age | | | Aₛ | L | Amin | D |
|---|---|---|---|---|---|---|---|
| Subject | (months) | Sex | V (mm³) | (mm²) | (mm) | (mm²) | (mm) |
| 15 | 9 | F | 8034 | 7154 | 113.4 | 31.8 | 1.12 |
| 2 | 3 | M | 13430 | 8723 | 95.6 | 65.2 | 1.54 |
| 3 | 3 | M | 11924 | 8646 | 100.6 | 64.9 | 1.38 |
| 4 | 4 | F | 7913 | 6760 | 98 | 45.1 | 1.17 |
| 5 | 5 | F | 8599 | 7573 | 89.9 | 66.6 | 1.14 |
| 6 | 6 | M | 8723 | 9641 | 110.3 | 58.7 | 0.905 |
| 7 | 7 | M | 11789 | 10241 | 109.7 | 54.2 | 1.15 |
| 8 | 8 | M | 10342 | 10142 | 110 | 73.5 | 1.02 |
| 10 | 16 | M | 18583 | 13693 | 111.3 | 62.3 | 1.36 |
| 11 | 18 | F | 10754 | 9700 | 100.6 | 84.1 | 1.11 |
| 14 | 15 | M | 14304 | 11331 | 115.3 | 47.9 | 1.26 |

Parameters of each subject, such as the volume of the airway, the area of the surface lumen of the airway, the length of a representative path through the airway and the minimum cross-sectional area of the airway were measured from CT scans of the built replicas and are reported in Table 1 along with the ages and genders of each subject.

In order to allow easy connection of the airway models to downstream tubing, each airway was appended with a distal 2 cm right prismatic extension.

The pressure drop across the airway of each model was measured for a number of steady flow rates as an independent experiment. Flow was measured with a mass flow meter (4143 series, TSI, Shoreview, Minn.). Pressure drop was measured with a differential pressure meter (HHP-103, Omega, Stamford, Conn.).

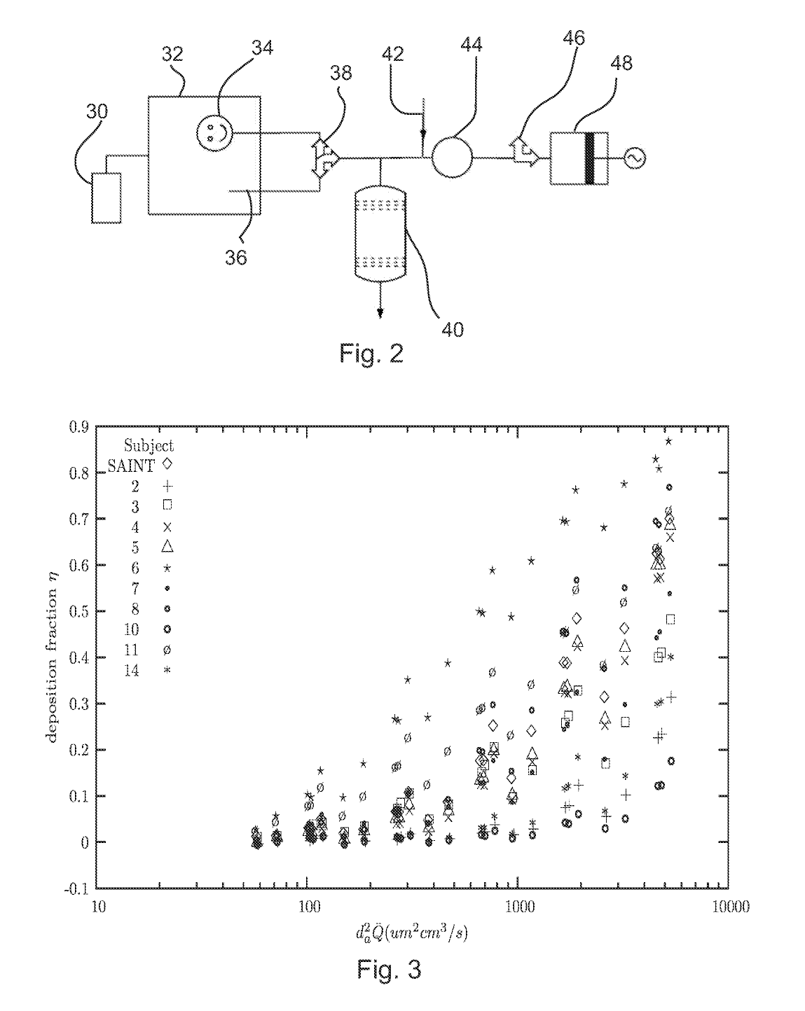

A number of experiments to measure nasal deposition were carried out using the setup as shown in FIG. 2. From upstream to downstream the setup consisted of the following:

A six jet collision nebulizer 30 (BGI, Inc. Waltham, Mass.) connected to filtered house air producing a polydisperse aerosol of sunflower oil (ρ=0.92 g/mL) with particles ranging in aerodynamic diameter from 0.8-5.3 μm. However, particles from 0.1-10 μm may be used.

A chamber 32, equipped with a mixing fan (not shown), housing the airway model 34 and a blank sampling line 36 of matched length.

A three way valve 38 to switch between the model 34 and the blank sampling line 36.

An electronic low pressure impactor 40 (ELPI) (Dekati Ltd., Tampere, Finland).

A filtered air-supply 42 matched to the flow volume through the ELPI.

A mass flow meter 44 (4143 series, TSI) measuring breathing flow.

A one-way valve 46 allowing only inhalation flow through the airway model 34.

A cyclic breathing machine 48, constructed in house, producing a sine wave breathing pattern. Flows through the ELPI are fixed at 30 L/min by critical flow at the outlet of the impactor. A filtered air-supply set to 30 L/min supplies the flow consumed by the ELPI through a relatively high resistance path. The cyclic breathing machine applies a varying pressure which results in flow primarily through the low resistance path which is either the sample line or nasal model depending on the position of the three-way valve.

Deposition was measured by comparing the amount of aerosol that passed through the blank sample line 36 to the amount that passed through the nasal model 34. No separate characterization of inhalability was performed and all measurements of deposition indicate the full fraction that did not pass through the model. Aerosol entered the replica 34 naturally from the air surrounding the facial features of the replicas, so that the present results include both inhalability and internal nasal deposition, although inhalability is close to 100% for the flow rates and particle sizes considered here.

A single deposition experiment consisted of a two minute sample drawn through the blank line 36 followed by a two minute sample through the model 34, and ended with another two minute sample through the blank line 36. Each two minute time interval was further divided with a forty second period at the beginning of the interval and a five second period at the end of each interval both being discarded to eliminate effects from settling of the instrument and errors in valve switching synchronization. This left a seventy-five second period of data which was actually used in each two minute interval. This was sufficient to allow averaging of many breathing cycles. The ELPI 40 provided an average particle count for size ranges corresponding to each stage of the impactor for each time interval. Nasal deposition for each impactor stage size range was calculated as the difference between the average number seen through the blank line 36 at that stage and the average number seen through the model 34 at that stage divided by the average number through the blank line 36 at that stage.

Five repeats of this experiment were performed for each model 34 shown in FIGS. 1A-1K using each of five breathing patterns and the results for each breathing pattern and model combination were individually averaged.

A small number of validation runs were performed with an electrostatic neutralizer upstream of the model and compared to runs made without the neutralizer. The two differed negligibly in measured deposition in the models, confirming that electrostatic effects were insignificant.

The breathing patterns used were half sinusoidal with only the inhalation portion of the curve being used. This was done to avoid many technical difficulties associated with using both the inhale and exhale flows given the small tidal volumes involved. In particular, the dead-space introduced by any length of sampling line would easily exceed the small tidal volumes being used, making proper sampling impossible. Balancing the volume of the model lines and sampling lines is also less critical if only the inhale flow is used. During in vivo breathing, there is a period of zero flow as exhalation stops and inhalation starts. The initial condition for inhalation is zero flow in both real breathing and in our patterns so it is not expected that using only the inhale flow will have had any effect on deposition. The typical parameters for the breathing patterns are given in Table 2 below, while the parameters for each individual experiment were measured and used for all calculations. In table 2, Vt=tidal volume, bpm=breaths per minute, and Q̅=average flow rate during inhalation.

TABLE 2
Breathing patterns used in the study.

| Vt | | |
|---|---|---|
| (cm 3) | bpm | Q̅ |
| 60.9 | 45 | 91.3 |
| 113.8 | 30 | 113.8 |
| 107.3 | 45 | 161.0 |
| 87.9 | 57 | 167.1 |
| 186.2 | 30 | 186.2 |

Average weight for the infants in this study ranged from 6 kg to 11 kg based on age and gender. Resting tidal volume ranges are known to be from 5 mL/kg to 8 mL/kg in infants and children. This gives a resting tidal volume range of 30 mL to 88 mL for the present subjects. Resting breathing rate ranges from 44 bpm to 34 bpm with age from 2 months to 18 months. A study examined 23 children aged 8-10.5 yrs and found an increase in breathing rate of 97% from that at rest vs. maximum exercise for boys in this age group, while girls increased breathing rate on average by 79%. In the same study tidal volume was found to increase 185% for boys and 135% for girls under maximum exercise. The largest tidal volume used in the present study was a 112% increase from resting volume while the highest breathing rate was a 26% increase from resting range. Extrapolating from this, the breathing patterns used in the present study represent states of moderate activity.

All curve fitting was done using Levenberg-Marquardt nonlinear regression with a least squares objective function in the GNU Octave software package version 2.1.72 (www.octave.org). Multiple starting points in the parameter space were used to ensure a reasonably global solution.

Deposition for all subjects versus impaction parameter, dₐ² Q̅, where dₐ is particle aerodynamic diameter, is shown in FIG. 3. A great deal of variability is evident across the subjects and deposition for any individual subject is not captured by a single curve.

Semi-empirical models to predict nasal deposition in individuals have been proposed based on pressure drop across the nose, maximum air velocity based on minimum airway cross-section area, and Stokes number scaled using the minimum airway cross-sectional area. These models have been based on in vivo and in vitro measurements.

A previous study of various adult oral airways found the data collapsed well using a correlation involving Reynolds and Stokes number based on mean cross-sectional area to predict aerosol deposition. In the present study subsequent results are presented as a function of Stokes or Stokes and Reynolds numbers in order to express deposition in a way that collapses the inter-subject variability using the appropriate non-dimensionalization. The Reynolds number is a ratio of the convective and viscous terms of the equations governing fluid motion and is the main parameter used to characterize incompressible flows. The Stokes number relates the stopping distance of a particle in the flow to a characteristic length scale in the geometry. This indicates a particle's ability to follow curves in the flow streamlines. A number of length scales were examined to non-dimensionalize the present infant deposition data. These included minimum cross-sectional area and average cross-sectional area. Trans-nasal pressure drop at 7˜L/min of flow was also tried as a scale factor by using the inverse of pressure drop in place of the length scale in the definition of Stokes and Reynolds numbers. Trans-nasal pressure drop may be used at other flow rates, although the flow rate may have to be similar to the flow rates at which the aerosol is to be inhaled. The length scale, D that best collapsed the data was found to be based upon a mean diameter of the nasal airway, for example airway volume divided by airway surface area. This can be seen by a comparison of r² values for fits using various scaling dimensions given in Table 3.

TABLE 3
r² comparison of fits using different length scales
D to define the Reynolds, Stoke, and the parametric correction.

| | Fit, D = | | | | | | | |
|---|---|---|---|---|---|---|---|---|
| | V/Aₛ | V/Aₘᵢₙ | L | Aₛ/L | \sqrt{V/L} | \sqrt{Aₘᵢₙ} | P | 1/P |
| Stk, Re | 0.887 | 0.745 | 0.545 | 0.606 | 0.792 | 0.495 | 0.351 | 0.753 |
| Stk, Re, D | 0.93 | 0.778 | 0.588 | 0.638 | 0.813 | 0.59 | 0.901 | 0.901 |

Hydraulic diameter is often used as a scale dimension in non-circular geometries and is defined as:

$${D}_{h} = \frac{4 * cross \text{-} sectional area}{perimeter}$$

By comparison, our chosen dimension D has some analogy to the hydraulic diameter for these geometries with variable cross-section since it is equivalent to:

$$D = \frac{average cross \text{-} sectional area}{average perimeter}$$

The average flow rate for each breathing pattern was used in the definition of Re and Stk. It was found that the flow pattern could be fully characterized, for our purposes, by average flow rate, with patterns having similar average flow rate but different combinations of Vt and breaths per minute resulting in similar deposition. The dynamics of the nasal flows are, of course, changing through the breathing cycle and deposition may be happening at different rates during the cycle and at different locations within the nasal geometry. Despite this, average flow rate was successfully used to create a correlation for total deposition.

A function of the form η=1−(a/(a+x))^{b}, where a and b are fit parameters was used to match the overall shape of the deposition curve. In order to capture the deposition for all subjects as a single curve, the abscissa was taken variously as a function of Stokes number; Stokes and Reynolds numbers; or Stokes number, Reynolds number and length scale D.

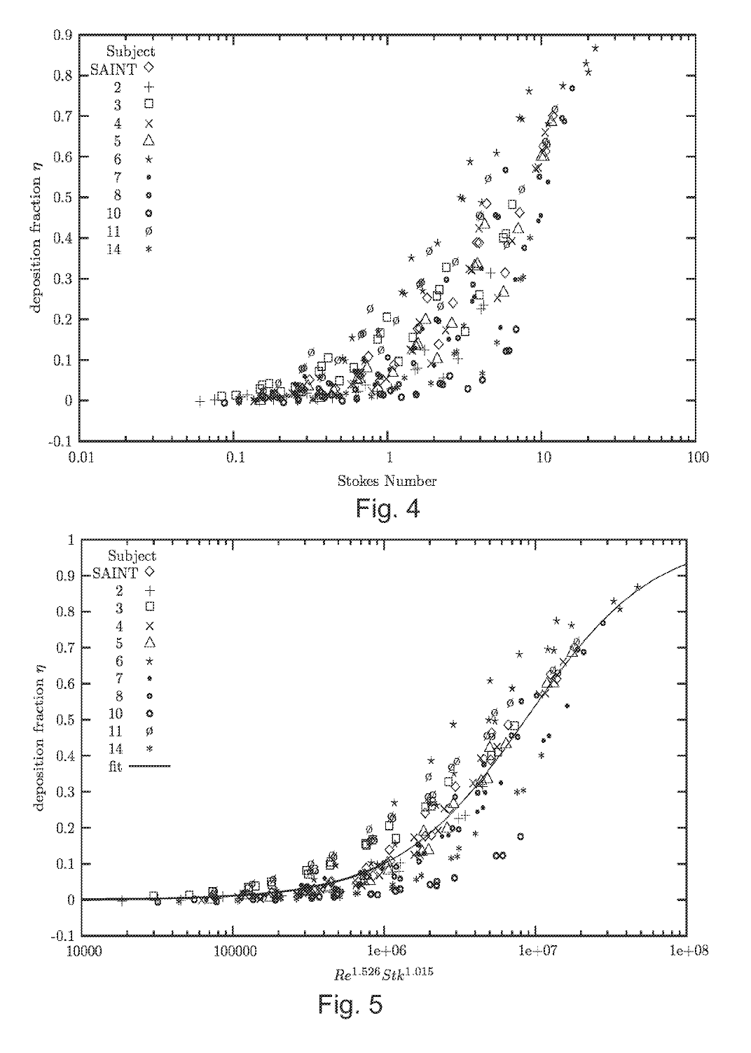

In one embodiment of the method, stage 304 further comprises generating nasal deposition prediction data based on a Stokes number that depends on a length scale D based on the geometrical properties of the nasal airway of the infant 50. Attempts to express deposition versus only Stokes number resulted in distinct deposition curves for each flow rate and each individual as seen in FIG. 4. The Stokes number (Stk) may be given by:

$$Stk = \frac{\overline{Q} {ρ}_{particle} {d}^{2} Cc}{18 μ {D}^{3}} \qquad ( 1 )$$

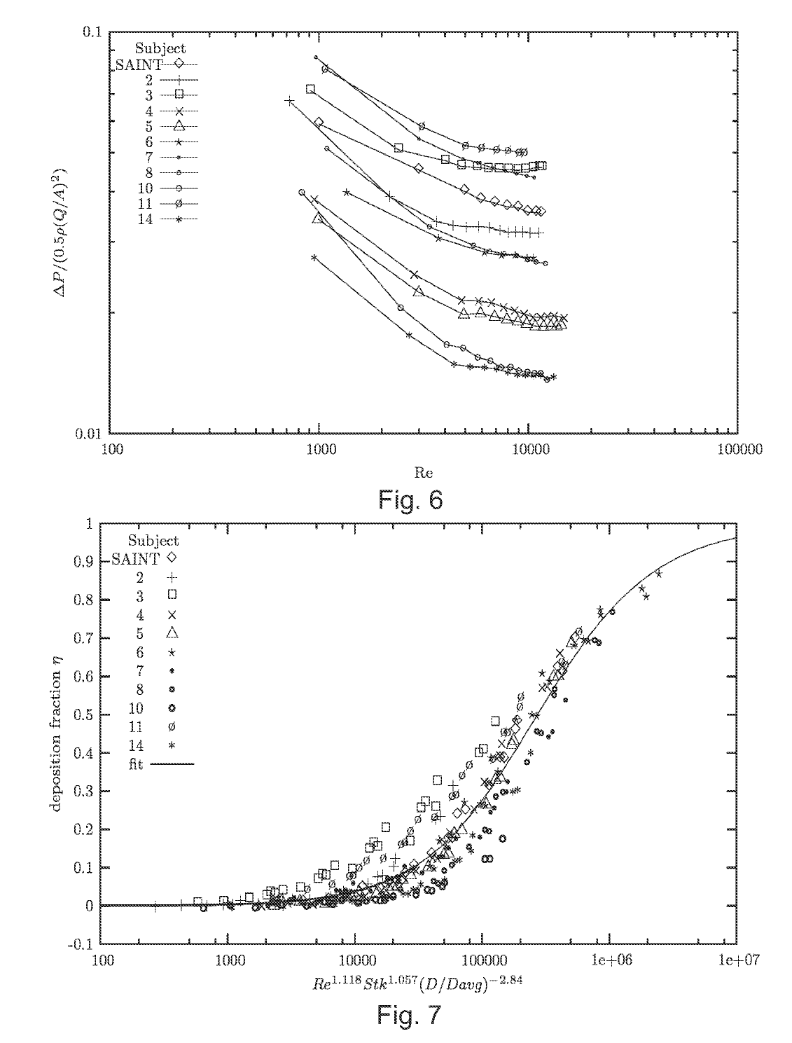

The results shown in FIG. 4 suggest an additional dependence on flow rate. A better fit is obtained if Reynolds number is also included as in FIG. 5. In some embodiments, the nasal deposition prediction data is further based on a Reynolds number that depends on a length scale D based on the geometrical properties of the nasal airway of the infant. The data illustrated in FIG. 5 is consistent with the observed static pressure drop for these models i.e. when non-dimensionalized pressure drop is plotted versus Reynolds number, as in FIG. 6, we see that non-dimensionalized pressure drop is not constant over the range of flow rates used. This suggests that the flow pattern is changing with flow rate over the range of flow rates used. The Reynolds number may be given by:

$$\begin{matrix}Re = \frac{\overline{Q} ρ}{D μ} & ( 2 ) \\ The fit function for deposition shown in FIG . 5 is \text{:} & \\ η = 1 - {( \frac{1.007 * {10}^{7}}{1.007 * {10}^{7} + ( {Re}^{1.526} {Stk}^{1.015} )} )}^{1.126} & ( 3 )\end{matrix}$$

Once η is determined, η may be used to predict the aerosol drug dose using the following formula or a functional equivalent:

$$w = \frac{z}{( 1 - η )} \qquad ( 4 )$$

where w is the aerosol drug dose and z is the amount of drug to be delivered to the lungs of the infant.

While the nasal geometries of each infant have similar overall shape, the details of each geometry differ, as seen in FIGS. 1A-1K. They are not scaled versions of the same structure so the use of the non-dimensional Stokes and Reynolds numbers is not expected to fully collapse the deposition data since it does not account for these individual differences. Thus, in some embodiments the nasal deposition prediction data is further based on a parametric correction that depends on a length scale D based on the geometrical properties of the nasal airway of the infant. This provides a parametric correction that includes Reynolds, Stokes and the geometric scale factor D, as shown in FIG. 7. This results in a further collapse of the data.

The fit function used in FIG. 7 is:

$$η = 1 - {( \frac{2.164 * {10}^{5}}{2.164 * {10}^{5} + ( {Re}^{1.118} {{Stk}^{1.057} ( D / {D}_{avg} )}^{- 2.840} )} )}^{0.8510} \qquad ( 5 )$$

where D_{avg}=1.20 mm for the group of infants tested. Equation 5 allows quantitative prediction of nasal deposition in individual infant subjects. Equation 5 illustrates nasal prediction data used to predict the fraction (η) of nasal airway deposition of the aerosol drug in this case.

Comparing infant nasal deposition from Eq. 5 to that seen in adults (Cheng, Y. S. (2003), “Aerosol deposition in the extrathoracic region”, Aerosol Science and Technology, 37, 659-671, hereinafter “Cheng”) for subjects at rest we find less nasal deposition in infants, e.g. for d=2 μm, Q̅=72 cm³ (4.3 L/min, which is a typical resting flow rate for subjects in the age range of infants considered here), D=D_{avg} and ρ_{particle}=1000 kg/m³, Eq. (5) gives η=2.0% for an infant, while the nasal deposition correlation of Cheng gives η=18.2% (assuming a resting adult flow rate of 15 L/min and taking average Aₘᵢₙ of 2.08 cm² as given). Much of this difference is attributable to the difference in resting flow rates.

Equations 3 and 5 were calculated with the length scale D of the Stokes number, the length scale D of the Reynolds number, and the length scale D of the parametric correction all based upon a mean diameter of the nasal airway, in this case D=(V/Aₛ). However, in some embodiments one or more of the length scale D of the Stokes number, the length scale D of the Reynolds number, and the length scale D of the parametric correction is based upon a mean diameter of the nasal airway. Differing equations will result, which are within the scope of this document.

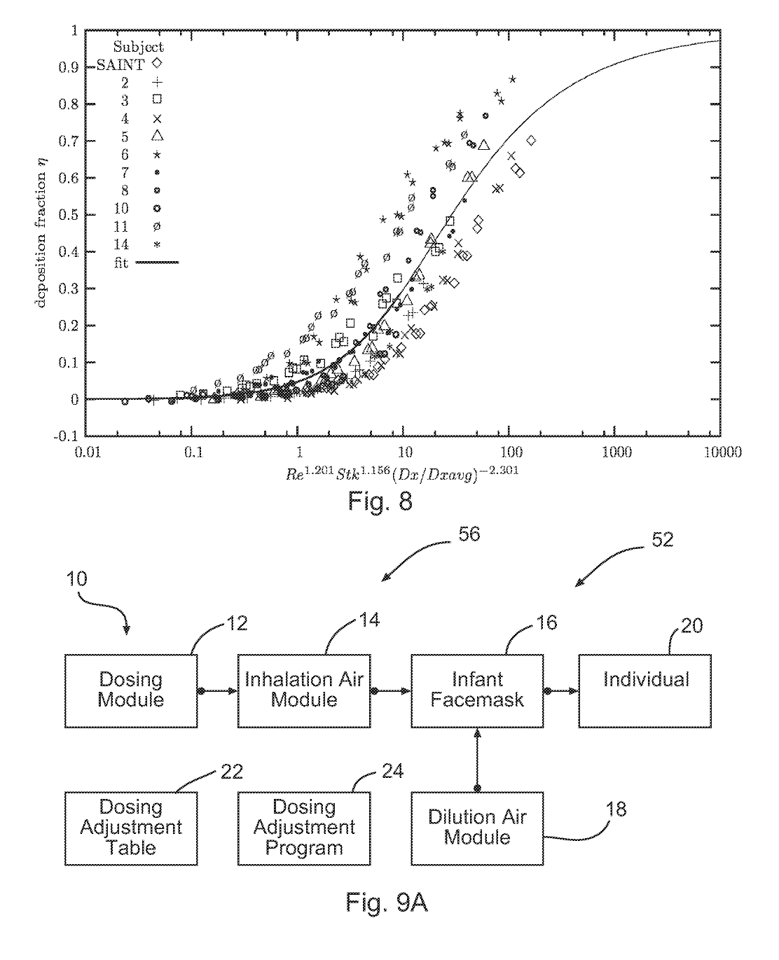

In some embodiments, one or more of the length scale D of the Stokes number, the length scale D of the Reynolds number, and the length scale D of the parametric correction is based upon a mean cross sectional area of the nasal airway. This may provide a useful correlation for cases where the average cross-sectional area is known, perhaps through acoustic rhinometry, while the airway surface area is not known. FIG. 8 gives a parametric fit using Reynolds number, Stokes number, and a parametric correction instead defined using an average cross-sectional length scale Dₓ with the additional term (parametric correction) depending directly on Dₓ, where Dₓ is defined as \sqrt{V/L}. Here V is the nasal airway volume and L is a nasal airway path length, for example the average nasal airway path length.

The fit function shown in Fig. is:

$$η = 1 - {( \frac{10.40}{10.40 + ( {Re}^{1.201} {{Stk}^{1.156} ( {D}_{x} / {D}_{x avg} )}^{- 2.301} )} )}^{0.5201} \qquad ( 6 )$$

where D_{xavg}=10.3 mm for the group tested. Equation 6 illustrates nasal prediction data used to predict the fraction (η) of nasal airway deposition of the aerosol drug.

This correlation also reveals some differences between oral geometries studied in adults and the nasal geometries examined in the current study. While an excellent collapse of oral deposition in adults has been obtained by others using only Reynolds number and Stokes number based on \sqrt{V/L}, FIGS. 7 and 8 show that the quantity V/Aₛ provides better collapse of variability for the infant nasal geometries studied. Also in contrast, we find here that a further dependence on a geometric factor beyond Reynolds number and Stokes number to obtain a tight collapse of the deposition data.

These differences may be explained by two factors. First the oral geometry is more circular in cross-section than the nasal geometry and therefore the square root of the average cross-sectional area is closer to the hydraulic diameter than is the case in the nasal geometry. Secondly, the oral geometries, being simpler, are apparently more easily modelled via the Reynolds number and Stokes number alone since their differences can be more accurately described by a volumetric scaling factor.

Referring to Table 3, examples are given of the various length scales D that may be used with the embodiments disclosed herein. In one embodiment, one or more of the length scale D of the Stokes number, the length scale D of the Reynolds number, and the length scale D of the parametric correction is based upon the trans-nasal pressure drop of the nasal airway. An exemplary length scale D of this type may be D=P, or D=1/P, which both give R² values of over 0.9. The length scale D may be dimensionless or dimensioned. One way to make D dimensionless when based on P is to define a velocity v= Q̅/A where Q̅ is the average inhalation flow rate and A is a geometrically based area, such as average cross sectional area, for the given nasal geometry. The parameter

$$\frac{P}{ρ {v}^{2}}$$

would then be dimensionless and may be used as D to create a best fit curve instead of using P alone.

In some embodiments, stage 304 may further comprise generating nasal deposition prediction data based on a Strouhal number (Sr) that depends on a length scale D based on the geometrical properties of the nasal airway of the infant. A suitable Strouhal number (Sr) may be used as a dimensionless parameter. The Strouhal number may be used either in addition to the Reynolds and Stokes numbers or instead of one of them. Parametric correction may also be carried out to tighten the fit. The Strouhal number captures unsteady effects due to the oscillatory nature of breathing. Sr may be defined as

$$Sr = \frac{fD}{v}$$

where D is a geometric length scale, f is a frequency related to breathing such as breathing frequency, and v is a flow velocity such as Q̅/A. Again, the length scale D may be varied in a similar fashion as the other length scales D discussed herein.

As indicated herein, the nasal deposition prediction data may be further based on one or more of: particle density of the dose, particle size of the dose, average inhalation flow rate of the dose, viscosity of the gas transporting the dose, and density of gas transporting the dose. Other variables may be taken into consideration.

Our measurements of nasal deposition in replicas of infants indicate that there is considerable variability in nasal aerosol deposition between individual infants. Deposition within a single subject may depend on Stokes number, and also on Stokes number and Reynolds number. This is apparent from our deposition data and consistent with our static pressure drop measurements.

A predictive equation for deposition in all eleven subjects was obtained by including both Stokes and Reynolds numbers scaled using total nasal air volume divided by surface area. An even tighter fit was obtained by including an additional direct dependence on volume over surface area. The resulting Eq. 5 allows estimation of nasal deposition in individual infants and is expected to be useful for those wanting to estimate the fraction of aerosol reaching the lungs through the nasal passage of infants during breathing. Thus a measure of extra-thoracic filtration of the aerosol drug dose is predicted.

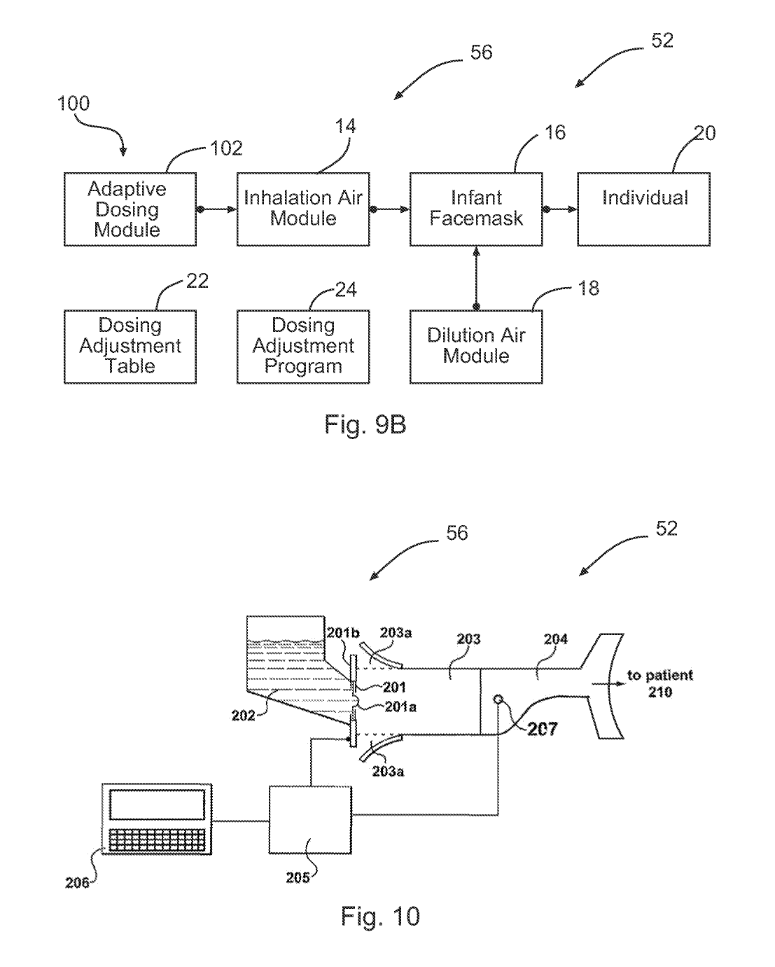

Referring to FIGS. 9A, 9B, and 10, various embodiments of suitable delivery systems are possible. It should be understood that in all embodiments of the delivery systems the aerosol drug dose may be based on the nasal deposition prediction data disclosed herein.

In one embodiment a conventional aerosol-based infant drug delivery system, such as an infant inhaler, may be produced that is configured to deliver the aerosol drug dose to the infant. Thus, the infant inhaler may provide a dose so that the desired amount of the aerosol drug reaches the lungs of the infant in use. Referring now to FIG. 9A, shown therein is block diagram of an exemplary inhaler 10 (or in more general terms, a delivery system). The inhaler 10 includes a dosing system 52, which may itself comprise a dosing module 12 and an inhalation air module 14. Inhaler 10 may also have an infant facemask 16. A dilution air module 18 may also be present. In other embodiments, the inhaler 10 may contain other modules (or leave out or exchange some of these modules) as is known to those skilled in the art. The loading/dosing module 12 provides a certain dose of an aerosol drug to an infant 20 who uses the inhaler 10. The inhalation air module 14 is responsible for mixing the aerosol drug with the air that is inhaled by the individual 20 when the infant facemask 16 is positioned over the infant's face during use. The dilution air module 18 provides an additional volume of air to the individual 20 to ensure that the aerosol drug is suitably diluted and inhaled at an adequate flow rate so that the aerosol drug reaches the lungs of the individual 20 in an appropriate fashion.

In some embodiments, a medical practitioner may prescribe a dosage for the dosing module 12 based on the geometrical properties of the infant, such as the information required to calculate the length scales D referred to herein. The required geometrical information may be obtained by a suitable method such as, but not limited to CT, Cone Beam Computed Tomography, X-ray, fluoroscopy, ultrasound, PET, or gamma scintigraphy. The medical practitioner may then refer to one or more of a dose adjustment table 22 or a dose adjustment computer program 24 that both comprise nasal deposition prediction data, and that can be used to predict nasal airway deposition according to the embodiments disclosed herein. The computer program may determine the aerosol drug dose based on the output from the diagnostic module 54. In general, any suitable means can be used to predict the amount of the aerosol drug dose according to the embodiments disclosed herein such that the correct or desired amount of medication reaches the lungs of the infant 20.

A suitable table 22 may involve the nasal airway volume and surface area as input parameters, and may use an average inhalation flow rate and particle size that occurs when a particular inhaler device is used. Accordingly, the table may be generated by using the equations specified above, while varying the values for V and Aₛ and assuming general or average values for the other parameters. Another realization of a table may include the specific infant's inhalation flow rate on the device as another input into the table. The infant's inhalation flow rate with the inhaler device may be measured using a suitable flow meter or spirometer. When including inhalation flow rate as an input parameter, it is also possible to embed into the tabular data flow rate dependent particle size information for the particular inhaler. There may be a set of tables, with each table being defined for a particular different set of variables. It is also possible to include in the table, or generate a set of tables based on, any parameter appearing in the Stokes and Reynolds numbers in the nasal deposition prediction data disclosed herein. This functionality may be incorporated into the adaptive dosing module of the inhaler 100, shown in FIG. 9B

Based on the prediction information, the medical practitioner is confident that an adjusted dosage is provided by the dosing system. For instance, if it is desired to deliver 40 mcg to the lungs of the infant 20 and the table 22 or the program 24 indicates that the infant has 50% deposition in the nasal airway region, then the medical practitioner can ensure that a dose of 80 mcg is provided to the infant facemask 16 by the dosing module 12 for inspiration by the infant 20. The aerosol drug dose is the dose that the inhaler 10 delivers to the infant 20. Alternatively, the predicted deposition data can be in terms of the loaded dose at any point in the process, such as after aerosolization but before inhalation air is added, by adding a suitable conversion factor, as is known by those skilled in the art, between the loaded and the delivered dose. The inhaler 10 may be loaded with a fixed dose for each inhalation, but the fixed dose may come in different predetermined amounts and each infant 20 is then prescribed the inhaler 10 that is loaded with the appropriate one of the predetermined amounts.

Referring now to FIG. 9B, shown therein is a block diagram of another exemplary embodiment of a delivery system, such as an inhaler 100, in accordance with the present disclosure. The inhaler 100 is similar to the inhaler 10 with the exception that the inhaler 100 includes an adaptive dosing module 102. The adaptive dosing module 102 allows the dosage of the aerosol drug delivered to the infant 20 to be tailored to the infant 20 so that the amount of the aerosol drug delivered to the lungs of the infant 20 is the desired dosage. In a similar fashion seen for the inhaler 10, for the inhaler 100, the medical practitioner can set the dose on the inhaler 100 by using nasal airway geometrical data and table 22 or program 24.

The adaptive dosing module 102 may include a drug reservoir from which the prescribed dose is loaded each time the patient inhales. The amount of the drug taken from the reservoir every time a dose is delivered is determined by the predictive deposition data obtained in accordance with the embodiments disclosed herein. A setting on the adaptive dosing module 102 may be adjusted by an operator, or another suitable mechanism, for this purpose. Alternatively, the adaptive dosing module 102 may be implemented such that it can receive drugs that are prepackaged in a number of different doses such as 50, 100, 200, 400 and 800 mcg doses or 50, 100, 150, 200, 300 and 500 mcg doses, or some other suitable sequence for example. Then, when prescribing the correct dosage, the medical practitioner may select the correct dose based on the volume and surface area of the nasal airway region of the infant and the table 22 or the computer program 24.

There are other ways to tailor the amount of aerosol drug, or the fashion in which the aerosol drug is provided to the infant 20 to ensure that the correct amount of medication reaches the lungs of the infant. For instance, in a further embodiment, the adaptive dosing module 102 may have a number of drug reservoirs in which the particle sizes of the different drug reservoirs is varied. Then the methods disclosed herein may be used to select a particle size to achieve a certain deposition in the lungs according to the deposition prediction data used. The particle size is chosen that results in the right fraction of aerosol-drug getting past the nasal airway region to deliver a specific dose into the lungs of the infant 20 when the inhaler 100 is loaded with a fixed dose.

In another embodiment, the inhalation flow rate may be varied instead of the particle size. In this case, the inhalation air module 14 may be replaced with an adaptive inhalation air module such that, given a certain dose in the inhaler and a certain desired lung dose, the nasal airway deposition prediction data may be used to select an inhalation flow rate that would deliver the right fraction of drug past the nasal airway region to deliver a specific dose into the lungs of the infant 20.

In a more general sense, any of the parameters in the Stokes number or Reynolds number may be used to predict that a desired fraction of the aerosol drug makes it past the nasal airway region and into the lungs of the infant 20. The parameters in the Stokes number that determine drug deposition: particle density, particle size, inhalation flow rate or velocity of particles in the nasal airway, viscosity of the inhaled gas (usually air), and nasal airway geometric dimensions. An additional parameter in the Reynolds number is gas density.

The predictive deposition equations may assume an average flow rate and average particle size for the inhaler. However, if more accuracy is desired, then the infant's inhalation flow rate through the inhaler can be measured and used in the predictive deposition equation as well, rather than relying on an average population value for inhalation flow rate. The infant's flow rate may be measured using a sensor, such as a fluid flow measurement device that is separate or could be added on to the inhaler.

Referring now to FIG. 10, another embodiment is illustrated. A delivery system, such as an inhaler 200, comprises a dosing system 52 and an infant facemask 204. A controller 205 may be connected to control the dosing system 52 for adjustment of the aerosol drug dose based upon the output of the diagnostic module 54 (shown in FIG. 11). An aerosol generator 201 for generating an aerosol and a reservoir 202 for storing the drug, for example in liquid form, that is supplied to the aerosol generator 201 to being nebulized when the aerosol generator 201 is operated. The controller may be connected to control the operation of the aerosol generator 201. The aerosol generator 201 may comprise a membrane 201a and an actuator 201b that is electrically controllable to actuate the membrane 201a in such a way that the membrane oscillates to produce an aerosol from the liquid supplied to the membrane. The aerosol generated by the aerosol generator 201 may be delivered to a mixing chamber 203 to allow air to be mixed with the aerosol so that the air carrying the nebulized liquid, i.e. the aerosol, may be inhaled by a patient 210 as the aerosol drug dose. The mixing chamber 203 comprises inlet openings, which may be inlet valves 203a, as shown in FIG. 10, for providing a passageway for ambient air to enter the mixing chamber 203 and to propagate to infant facemask 204 that is provided to facilitate inhalation through the inhaler 200 during an inhalation therapy.

As shown in FIG. 10, the delivery system 200 may comprise a controller 205 for controlling the operation of the aerosol generator 201 and therefore the generation of the aerosol. The controller may further comprise an input 206 for inputting the output of the diagnostic module 54, which may be processed and may include simply the required geometrical properties, to the controller 205. Input 206 may be connected to the controller 205 to allow an operator such as a medical practitioner to enter data describing morphological characteristics of the infant, i.e. the volume and the surface area of the patient's nasal airway region. This information is obtained by suitable means such as the embodiments disclosed herein. The controller 205 may be adapted to produce the nasal deposition prediction data as mentioned herein on the basis of the input parameters as well as an average inhalation flow rate and particle size to determine the delivered dose to the patient and to operate the aerosol generator 201 such that the correct or desired amount of medication reaches the lungs of the infant 210.

In another embodiment of a delivery system according to the embodiments disclosed herein, the delivery system may comprise a sensor 207 connected to supply measurement results indicating the infant's inhalation flow rate to the controller 205. The sensor 207 may be provided in or in the vicinity of the infant facemask 204. The measurement results outputted by the sensor 207 may be supplied to the controller 205 and the controller 205 may take the measurement results into account when controlling the operation of the aerosol generator 201. By taking into account the actual flow rate, a higher accuracy regarding the delivered dose may be achieved. The controller may be adapted to control the delivery device to vary at least one or more of: particle density of the aerosol drug dose, particle size of the aerosol drug dose, inhalation flow rate of the delivered drug, viscosity of the delivered dose, and gas density of the delivered dose, to deliver the aerosol drug dose.

Various geometrical properties of the nasal airway may be estimated or calculated from obtained data. The various constants and exponents of the provided equations are expected to vary depending on the body of data that the equations are derived from, and thus variability of these numbers is understood to be within the scope of the embodiments disclosed herein. Infants within the scope of this document refer to individuals from 0 months to three years of age. However, for the correlations presented herein, extrapolation errors may need to be accounted for in going outside the age range tested.

In the claims, the word “comprising” is used in its inclusive sense and does not exclude other elements being present. The indefinite article “a” before a claim feature does not exclude more than one of the feature being present. Each one of the individual features described here may be used in one or more embodiments and is not, by virtue only of being described here, to be construed as essential to all embodiments as defined by the claims.

1. A method of determining the dose of an aerosol drug to be delivered to an infant comprising:
a) determining a desired amount of the aerosol drug to be delivered to the lungs of the infant;
b) obtaining values for the geometrical properties of the nasal airway of the infant; and
c) determining a dose of the aerosol drug, according to the geometrical properties of the nasal airway, that will deliver the desired amount of the aerosol drug to the lungs of the infant.

2. The method of claim 1, in which step c) further comprises generating nasal deposition prediction data based on a Stokes number that depends on a length scale D based on the geometrical properties of the nasal airway of the infant.

3. The method of claim 2 in which the Stokes number (Stk) is given by:
$$Stk = \frac{\overline{Q} {ρ}_{particle} {d}^{2} Cc}{18 μ {D}^{3}}$$
where Q̅ is the average volumetric flow rate, ρ_{particle} is particle density of the aerosol drug, d is the particle diameter of the aerosol drug, CC is the Cunningham slip factor, μ is the fluid dynamic viscosity of the delivered dose, and D is the length scale D of the Stokes number.

4. The method of claim 2 in which the nasal deposition prediction data is further based on a Reynolds number that depends on a length scale D based on the geometrical properties of the nasal airway of the infant.

5. The method of claim 4 in which the Reynolds number (Re) is given by:
$$Re = \frac{\overline{Q} ρ}{D μ}$$
where Q̅ is the average volumetric flow rate, ρ is the fluid density of the aerosol drug, μ is fluid dynamic viscosity of the delivered dose, and D is the length scale D of the Reynolds number.

6. The method of claim 4 in which the nasal airway deposition prediction data predicts a fraction (η) of nasal airway deposition of the aerosol drug, the nasal deposition prediction data being defined by:
$$η = 1 - {( \frac{1.007 * {10}^{7}}{1.007 * {10}^{7} + ( {Re}^{1.526} {Stk}^{1.015} )} )}^{1.126}$$
where Stk is the Stokes number and Re is the Reynolds number.

7. The method of claim 4 in which the nasal deposition prediction data is further based on a parametric correction that depends on a length scale D based on the geometrical properties of the nasal airway of the infant.

8. The method of claim 2 in which one or more of the length scale D of which one or more of the length scale D of the Stokes number, the length scale D of the Reynolds number, and the length scale D of the parametric correction is based upon a mean diameter of the nasal airway.

9. The method of claim 8 in which one or more of the length scale D of the Stokes number, the length scale D of the Reynolds number, and the length scale D of the parametric correction are defined by:
D=(V/Aₛ)
where V is the volume of the nasal airway of the infant and Aₛ is the surface area of the nasal airway of the infant.

10. The method of claim 2 in which:
the nasal deposition prediction data is further based on a Reynolds number (Re) that depends on a length scale D based on the geometrical properties of the nasal airway of the infant;
the nasal deposition prediction data is further based on a parametric correction that depends on a length scale D based on the geometrical properties of the nasal airway of the infant; and
in which the length scale D of the Stokes number, the length scale D of the Reynolds number, and the length scale D of the parametric correction are defined by:
D=(V/Aₛ)
where V is the volume of the nasal airway of the infant and Aₛ is the surface area of the nasal airway of the infant.

11. The method of claim 10 in which the nasal deposition prediction data predicts a fraction (η) of nasal airway deposition of the aerosol drug, the nasal deposition prediction data being defined by:
$$η = 1 - {( \frac{2.164 * {10}^{5}}{2.164 * {10}^{5} + ( {Re}^{1.118} {{Stk}^{1.057} ( D / {D}_{avg} )}^{- 2.840} )} )}^{0.8510}$$
where Stk is the Stokes number, Re is the Reynolds number, D is the length scale, and D_{avg} is the average length scale D from a representative group of infants.

12. The method of claim 2 in which one or more of the length scale D of the Stokes number, the length scale D of the Reynolds number, and the length scale D of the parametric correction is based upon a mean cross sectional area of the nasal airway.

13. The method of claim 12 in which one or more of the length scale D of the Stokes number, the length scale D of the Reynolds number, and the length scale D of the parametric correction is defined by:
D=(V/L)̂0.5
where V is the volume of the nasal airway of the infant and L is a nasal airway path length.

14. The method of claim 2 in which:
the nasal deposition prediction data is further based on a Reynolds number (Re) that depends on a length scale D based on the geometrical properties of the nasal airway of the infant;
the nasal deposition prediction data is further based on a parametric correction that depends on a length scale D based on the geometrical properties of the nasal airway of the infant; and
the length scale D of the Stokes number, the length scale D of the Reynolds number, and the length scale D of the parametric correction are defined by:
D=(V/L)̂0.5
where V is the volume of the nasal airway of the infant and L is a nasal airway path length.

15. The method of claim 14 in which the nasal deposition prediction data predicts a fraction (η) of nasal airway deposition of the aerosol drug, the nasal deposition prediction data being defined by:
$$η = 1 - {( \frac{10.35}{10.35 + ( {Re}^{1.201} {{Stk}^{1.156} ( {D}_{x} / {D}_{x avg} )}^{- 2.301} )} )}^{0.5203}$$
where Stk is the Stokes number, Re is the Reynolds number, D is the length scale, and D_{avg} is the average length scale D from a representative group of infants.

16. The method of claim 2 in which one or more of the length scale D of the Stokes number, the length scale D of the Reynolds number, and the length scale D of the parametric correction is based upon the trans-nasal pressure drop of the nasal airway.

17. The method of claim 16 in which one or more of the length scale D of the Stokes number, the length scale D of the Reynolds number, and the length scale D of the parametric correction is defined by:
D=P
where P is the trans-nasal pressure drop.

18. The method of claim 16 in which one or more of the length scale D of the Stokes number, the length scale D of the Reynolds number, and the length scale D of the parametric correction is defined by:
D=1/P
where P is the trans-nasal pressure drop.

19. The method of claim 2 in which the nasal deposition prediction data is further based on one or more of: particle density of the dose, particle size of the dose, average inhalation flow rate of the dose, viscosity of the gas transporting the dose, and density of gas transporting the dose.

20. The method of claim 1 further comprising delivering the dose of the aerosol drug to the infant.

21. The method of claim 1 in which step c) further comprises generating nasal deposition prediction data based on a Strouhal number (Sr) that depends on a length scale D based on the geometrical properties of the nasal airway of the infant.

22. The method of claim 21 in which the Strouhal number (Sr) is defined by:
$$Sr = \frac{fD}{v}$$
where f is a frequency related to breathing and v is a flow velocity.

23. A delivery system for delivering an aerosol drug to an infant, the delivery device comprising:
a) a diagnostic module configured to provide geometrical properties of the nasal airway of the infant as output;
b) a dosing system configured to produce an aerosol drug dose, based upon the output of the diagnostic module, that is predicted to ensure that a desired amount of the aerosol drug dose reaches the lungs of the infant in use; and
c) an infant facemask connected to receive the aerosol drug dose from the dosing system for supply of the aerosol drug dose to the infant.

24-41. (canceled)
